(19) United States
(12) Patent Application Publication
(10) Pub. No.: US 2017/0202505 A1
(43) Pub. Date: Jul. 20, 2017

(54) UNOBTRUSIVE SKIN TISSUE HYDRATION DETERMINING DEVICE AND RELATED METHOD

(71) Applicant: KONINKLIJKE PHILIPS N.V., EINDHOVEN (NL)

(72) Inventors: Ihor Olehovych KIRENKO, VELDHOVEN (NL); Willem VERKRUIJSSE, VELDHOVEN (NL)

(21) Appl. No.: 15/326,807

(22) PCT Filed: Jul. 21, 2015
(86) PCT No.: PCT/EP2015/066688

§ 371 Date: Jan. 17, 2017

(30) Foreign Application Priority Data
Jul. 22, 2014 (EP) 14178042.9

(51) Int. Cl. A61B 5/00; A61B 5/024; A61B 5/1455

(52) U.S. Cl. CPC A61B 5/443; A61B 5/4875; A61B 5/14551; A61B 5/02416; A61B 5/7278

(57) ABSTRACT

The present disclosure relates to a method and a device (10) for determining a hydration state of skin tissue. The device (10) comprises an interface (22) for receiving a data stream (100) comprising a plurality of PPG signals including at least a first PPG signal (104) indicative of a first wavelength range (72), a second PPG signal (106) indicative of a second wavelength range (74), and a third PPG signal (108) indicative of a third wavelength range (76), a processing unit (24) for computing at least a first combined signal (116) based on a first pair of signals (104, 106) selected from the plurality of PPG signals, and a second combined signal (118) based on a second pair of signals (108, 110) selected from the plurality of PPG signals, wherein at least one PPG signal of the second combined signal (118) is dependent on a tissue hydration level, and an analysis unit (26) for computing a hydration signal (34) indicative of skin hydration, wherein the hydration signal (34) is derived from the first combined signal (116) and the second combined signal (118).

### FIELD OF THE INVENTION

The present invention relates to a device and a method for determining a hydration state of skin tissue. In particular, the present invention relates to an unobtrusive remote PPG monitoring device and to a related method that may be utilized so as to determine skin tissue hydration indicative signals. More generally, the present invention relates to unobtrusive (optical) non-contact measurement approaches which can be used for detecting physiological parameters in an observed subject. In this connection, optical measurement may particularly refer to photoplethysmography (PPG) and, to some extent, to pulse oximetry. The present invention further relates to a corresponding computer program.

### BACKGROUND OF THE INVENTION

US 2013/0210058 A1 discloses an apparatus for determining the hydration state of human tissue by near-infrared spectroscopy, the apparatus comprising an illuminator configured to illuminate the human tissue with near-infrared light; an optical receiver configured to receive near-infrared from the human tissue; a spectrometer in optical communication with the optical receiver, said spectrometer producing an output indicative of a spectrum; and a processing system configured to receive the output from the spectrometer and determine an output indicative of the hydration state in the human tissue. The document further discloses several refinements of the above apparatus.

In the field of health monitoring, skin hydration may be regarded as a key factor that is indicative of a current skin health status of an observed subject of interest (e.g., a patient). Skin hydration measurements may basically provide diagnostic information with respect to an actual health condition of the subject's skin. Furthermore, skin hydration measurements may indicate the integrity of the skin barrier function.

As used herein, skin hydration and skin tissue hydration may be used synonymously. With respect to the skin tissue, generally human skin tissue may be contemplated which, however, shall not be understood in a limiting manner. Generally, skin tissue hydration may be measured by means of near-infrared spectroscopy. As shown in US 2013/0210058 A1, conventional skin tissue hydration determining devices require near-infrared light sources and a spectrometer that is capable of illustrating and processing spectral information. In the field of skin tissue hydration measurement, conventional spectroscopy-based devices make use of the fact that an actual water content of living tissue typically influences the tissue reflectance at a particular wavelength.

However, spectroscopy-based hydration detection devices require considerably costly hardware and carefully control laboratory conditions. Consequently, spectroscopy-based skin tissue hydration measurement is not yet widely utilized. Furthermore, spectroscopy-based devices and methods basically require stable monitoring conditions which basically pose huge challenges for unobtrusive non-contact remote measurement approaches. By contrast, skin tissue hydration determination under constant laboratory conditions is typically experienced by patients as being unpleasant and uncomfortable.

In the field of unobtrusive remote non-contact patient monitoring, considerable progress has been made in recent years. In this respect, for instance, remote photoplethysmography (rPPG) has been presented. Generally, PPG-based methods monitor periodic color changes of the skin that are attributable to perfusion (pulsatile blood flow). Photoplethysmography, particularly remote photoplethysmography, may be utilized to monitor several vital signs, such as heart rate, heart rate variability, respiration rate, arterial blood oxygenation (oxygen saturation), etc.

Further, one or more video cameras are used for unobtrusively monitoring the heart rate (HR), the respiration rate (RR) or other vital signs of a subject by use of remote photoplethysmographic imaging. Remote photoplethysmographic imaging is, for instance, described in Wim Verkruysse, Lars O. Svaasand, and J. Stuart Nelson, “Remote plethysmographic imaging using ambient light”, Optics Express, Vol. 16, No. 26, December 2008. It is based on the principle that temporal variations in blood volume in the skin lead to variations in light absorptions by the skin. Such variations can be registered by a video camera that takes images of a skin area, e.g. the face, while the pixel average over a selected region (typically part of the cheek in this system) is calculated. By looking at periodic variations of this average signal, the heart rate and respiratory rate can be extracted. There are meanwhile a number of further publications and patent applications that describe details of devices and methods for obtaining vital signs of a patient by use of remote PPG.

Compared to spectroscopy-based methods, remote photoplethysmography-based methods and approaches generally require less expensive hardware. PPG-based imaging methods further distinguish over spectroscopy-based monitoring methods in that the latter ones are far more susceptible to disturbances, noise and further environmental influences, such as relative motion between an observed (skin) area and a respective sensor (imaging device, such as a camera). This is at least partially attributable to signal processing fundamentals inherent to the respective methods.

In spectroscopy-based methods, generally DC signal portions are monitored, measured and processed. In other words, a more or less constant absolute signal level is observed. Typically, disturbances that adversely affect the signal to noise ratio impact the DC portion of the observed signal. By contrast, PPG-based methods focus on AC signal portions that are superimpose on the DC portion. An AC signal portion may be regarded as a pulsatile signal portion that indicates more or less periodic changes of the signal level that are attributable to the pulsatile blood flow. In other words, overall disturbances may mainly corrupt DC signal portions while the AC signal portions may be generally unaffected. Consequently, PPG-based methods are much more suitable to remote monitoring and detecting approaches.

### SUMMARY OF THE INVENTION

It is therefore an object of the present invention to provide a device and a method for unobtrusively remote PPG monitoring for determining a hydration state of skin tissue that can make use of remote PPG implementation approaches and allow a cost-effective robust detection of skin tissue hydration levels with increased precision and reliability. It is further preferred that several drawbacks inherent to spectroscopy-based hydration detection devices may be overcome by the device and the method in accordance with the present disclosure. Preferably, the device and the method are particularly suited for unobtrusive remote non-contact monitoring.

In a first aspect of the present disclosure, an unobtrusive PPG monitoring device for determining a hydration state of skin tissue is presented, the device comprising:
an interface for receiving a data stream comprising a plurality of PPG signals including at least a first PPG signal indicative of a first wavelength range, a second PPG signal indicative of a second wavelength range, and a third PPG signal indicative of a third wavelength range,
a processing unit for computing at least a first combined signal based on a first pair of signals selected from the plurality of PPG signals, and a second combined signal based on a second pair of signals selected from the plurality of PPG signals, wherein at least one PPG signal of the first or second combined signal is dependent on a tissue hydration level, and the PPG signals of the other combined signal are not dependent on the tissue hydration level; and
an analysis unit for computing a hydration signal indicative of skin hydration, wherein the hydration signal is derived from the first combined signal and the second combined signal.

Preferably, but not to be understood in a limiting sense, the device is arranged as an unobtrusive remote PPG monitoring device. Consequently, the device may be arranged as a non-contact PPG monitoring device that is capable of processing a remotely detected data stream. As used herein, the term remotely detected data shall refer to sensors (e.g. video cameras, photodiodes, and suchlike) that are arranged at a distance from the subject of interest. Remotely detected data may involve video data, image data, etc. However, at least in some embodiments, the device can be arranged as a contact PPG monitoring device that comprises—or makes use of—contact PPG sensors.

The present invention is based on the insight that PPG monitoring approaches, particularly remote PPG monitoring approaches, may be utilized for the detection of skin tissue hydration information. More particularly, it has been observed that PPG signals which may be indicative of respective distinct wavelengths or wavelength ranges may be influenced by a present level of water deposition in the skin tissue. This may, on the one hand, adversely affect the precision of known PPG measurements, such as S_{P}O₂ measurements (oxygen saturation measurement), etc. However, the inventors concluded that, on the other hand, a dependency on an actual level of water deposition in the skin tissue may be utilized so as to obtain hydration state information from the respectively influenced PPG signals.

More specifically, the present invention is based on the assumption that the S_{P}O₂ values calculated from two pairs of PPG signals should be equal, unless one of the PPG signals (in the wavelength sensitive to hydration) is influenced by a different level of hydration. Thus, a S_{P}O₂ value calculated based on the two PPG signals, which wavelengths are not dependent on the hydration levels, can serve as a “reference” value to compare another S_{P}O₂ value calculated based on PPG signals, wherein at least one of the wavelength is sensitive to the skin hydration level. The difference between two S_{P}O₂ values is an indication of the hydration level.

Generally, water exhibits a characteristic absorption behavior for incident light (or, more generally, incident radiation) which is dependent on wavelength. Since also further “constituents” of skin tissue (hemoglobin, melanin, etc.) exhibit a respective characteristic absorption behavior, a plurality of PPG signals each of which is associated with a respective wavelength or wavelength range may be analyzed so as to draw hydration state-indicative information therefrom. In other words, a system of equations with multiple unknowns (e.g. the proportion of the constituents in the skin tissue) may be formed and solved under consideration of at least two combined signals that are based on selected PPG signals from the plurality of PPG signals.

To this end, radiation emitted or reflected by a subject can be captured and processed accordingly. As used herein, the term “radiation emitted or reflected by a subject” may generally refer to radiation which is emitted towards and eventually re-emitted by the subject of interest. For instance, incident radiation may be specularly reflected at the subject's skin surface. Furthermore, incident radiation may be diffusely reflected at subjacent portions of the subject's skin tissue. Still, however, incident radiation may also be transmitted through the subject's skin tissue, for instance at the fingertip or the earlobe. Transmission of radiation may involve direct transmission, but also deflected transmission. All these events may be covered by the term “re-emitted”. Typically, re-emitted radiation can be composed of several portions which may have been subjected to various types of reflection or transmission.

Generally, the data stream can comprise a sequence or a set of sequences of frames or, more precisely, a series or a set of series of image frames comprising spectral information based on a representation of a region of interest. As used herein, the data stream may be generally composed of image data, particularly video data. Consequently, the data stream may be composed of a consecutive series of video frames. The respective PPG signals can be drawn from the image data of which the data screen is composed. The data stream may comprise image data which is indicative of a considerably broad wavelength range. By way of example, the data stream may cover at least a fraction of visible light and a fraction of infrared light. As used herein, the term visible light shall refer to electromagnetic radiation that is visible to the human eye. In other words, visible light may span a range between about 390 nm (nanometer) to 700 nm. Adjacent to the visible light fraction of the electromagnetic spectrum, the infrared fraction is provided which may span a range of about 700 nm (nanometers) to about 1 mm (millimeters). The infrared portion of the electromagnetic spectrum may be divided into further sub-portions. By way of example, a respective near-infrared portion may cover a range of about 750 nm to about 1.4 μm (micrometers). A short-wavelength infrared portion may cover a range of about 1.4 μm to about 3 μm. A mid-wavelength infrared portion may cover a range of about 3 μm to about 8 μm, etc.

By way of example, each of the first PPG signal, second PPG signal, third PPG signal and, if any, fourth PPG signal, etc. may be indicative of a respective sub-portion or band in the read and infrared portion, for instance in the range between about 600 nm to about 1200 nm. It goes without saying that each of the ranges the respective PPG signals are indicative of, a considerably narrow sub-portion of the band, or in extreme cases of singular wavelength “lines”. The respective wavelength ranges the PPG signals are indicative of are preferably distinct from each other and clearly distinguishable (in terms of the absorption behavior of the skin tissue constituents). However, at least in some embodiments, at least some of the wavelength ranges may at least partially overlap.

Preferably, each of the plurality of PPG signals is at least partially attributable to pulsatile color changes due to time variant blood perfusion. Furthermore, preferably at least some signals of the plurality of the PPG signals are considerably sensitive to water deposition in the monitored skin tissue, while the other PPG signals are not sensitive to water deposition in the monitored skin tissue. The more water is deposited in the skin tissue, the more water absorption influences the PPG signal which is analyzed by the device. Since the water content basically differently influences the distinct wavelength ranges, respective distinct hydration-relative “offsets” may occur. Since a plurality of PPG signals, preferably at least three PPG signals, are selectively combined to derive at least a first pair of signals and a second pair of signals, the hydration-related signal offset may be identified, at least to some extent. Consequently, absolute and/or relative hydration-related signals may be detected accordingly.

The analysis unit is basically configured to perform a comparison between the first pair of signals and the second pair of signals. Assuming that the hydration state would have no impact on the PPG signals, both the first pair of signals and the second pair of signals would be basically equivalent (for instance in terms of a level of blood oxygenation). However, since water absorbs electromagnetic radiation to a varying degree, dependent on wavelength, different “signal offsets” at the first combined signal (based on the first pair of signals) and the second combined signal (based on the second pair of signals) may be expected. Having at least some knowledge of these “signal offsets” basically allows to conclude a level of hydration from the detected and processed PPG signals.

In one embodiment, the analysis unit is further configured to match the first combined signal and the second combined signal, and to compute the hydration signal based on the signal match. By way of example, each of the first combined signal and the second combined signal may comprise a respective ratio of the underlying PPG signals. Computing the first combined signal and the second combined signal may further involve applying calibration constants to the respective values. Assuming that a significant difference between the first combined signal and the second combined signal is detected, the respective calibration constants for, for instance, the second combined signal may be adapted so as to “match” the first combined signal and the second combined signal. Based on the required variation of the calibration constants, a hydration-indicative signal may be derived.

Consequently, the analysis unit may, in yet another embodiment, be further configured to compute the hydration signal under consideration of a predefined set of calibration constants for the first combined signal by revising variable calibration constants for the second combined signal.

In yet another embodiment, the analysis unit may be further configured to monitor relative changes of the hydration signal over time in a continuous or quasi-continuous manner. By way of example, an initial hydration level may be computed. Based on the respective initial values, variations between the first combined signal and the second combined signal may be tracked and monitored over time. This may allow conclusions as to a qualitative trend of the hydration state of the observed subject's skin tissue. Consequently, trend monitoring which may allow conclusions as to relative and/or absolute changes of the skin tissue hydration level may be enabled. Also qualitative trend monitoring can be envisaged.

In yet another embodiment, the processing unit may be further configured to compute the at least first combined signal under consideration of a first predefined set of calibration constants and to compute the at least second combined signal under consideration of a second predefined set of calibration constants, wherein the analysis unit is further configured to compute the hydration signal based on reference values for skin hydration levels and a detected discrepancy between the at least first combined signal and the at least second combined signal.

To this end, reference data may be provided, for instance, look-up tables, etc. As already indicated above, differences between the first combined signal and the second combined signal are basically indicative of a present tissue hydration level. In accordance with the above embodiment, no “signal match” of the first combined signal and the second combined signal by a respective variation of calibration constants is necessary. Based on the detected difference between the first combined signal and the second combined signal, the resulting skin hydration level may be obtained from the reference data. Reference data may be obtained/generated in upstream reference measurements to define a respective set or database of reference values and/or to derive a characteristic formula or an assignment algorithm (input value: detected difference; output value: hydration level). Reference data may be universally applicable overall reference data. However, at least in some embodiments, specific reference data may be obtained by applying reference measurements to the currently to-be-monitored subject. It may be preferred in this context to apply reference measurements on the basis of conventional spectroscopy-based skin tissue hydration measurements. Consequently, an absolute and/or relative measurement scale may be generated.

The above embodiment may be further developed in that the analysis unit is configured to monitor absolute tissue hydration level the use based on a data set of reference values obtained from reference measurements. Consequently, also spot measurement may be enabled.

In yet another embodiment of the device, the first wavelength range is selected from the red wavelength range, wherein the second wavelength range is selected from the near infrared wavelength range, wherein the third wavelength range is selected from a deeper infrared wavelength range, and wherein the third wavelength range comprises larger wavelengths than the second wavelength range. Preferably, water deposition in the skin tissue differently influences the absorption behavior of the skin tissue in the respective wavelength ranges.

The above embodiment may be further developed in that the plurality of PPG signals further comprises at least a fourth PPG signal indicative of a fourth wavelength range, wherein the fourth wavelength range is selected from a wavelength portion spanning the red wavelength band and the near infrared wavelength band. Preferably, the fourth wavelength range is selected from a deeper infrared wavelength range. Consequently, the first combined signal may be based on the first PPG signal and the second PPG signal, whereas the second combined signal may be based on the third PPG signal and the fourth PPG signal, for instance.

In yet another embodiment, each of the first combined signal and the second combined signal is an oxygen saturation indicative signal. This may have the advantage that the device may be configured, in an oxygen saturation detection mode, to determine oxygen saturation indicative signals and, in a skin hydration measurement mode, to determine hydration indicative signals. The oxygen saturation detection mode and the skin hydration measurement mode may be operated in parallel.

An important field for PPG measurements is the determination of blood oxygen saturation. In the field of pulse oximetry measurement, PPG-based devices are known that operate at two distinct wavelength bands, for instance in the red wavelength band and the infrared wavelength band. By way of example, conventional obtrusive contact PPG devices may be arranged as finger clips, ear clips, etc. Contact pulse oximeters typically transmit red and infrared (or, more precisely, in some cases near infrared) light through a vascular tissue of the subject of interest. The respective light portions (R/IR) can be transmitted and detected in an alternating (fast-switching) manner. Given that the respective spectral portions are differently absorbed by oxygenated hemoglobin (HbO₂) and reduced hemoglobin (Hb), blood oxygen saturation eventually can be processed. An oxygen saturation (SpO₂) estimation algorithm can make use of a ratio of the signals related to the red and the infrared portion. Furthermore, the algorithm can consider a non-pulsatile signal component. Typically, the PPG signal comprises a DC component and a relatively small pulsatile AC component. Furthermore, SpO₂ estimation generally involves an empirically derived calibration factor applied to the processed values. Typically, the calibration factor (or, calibration curve) is determined upon reference measurements involving invasive blood oxygen saturation measurements. A calibration factor is required since a PPG device basically detects a ratio of (spectral) signal portions which has to be transferred into a blood oxygen saturation value which typically involves a ratio of HbO₂ and Hb. For instance, but not intended to limit the present disclosure, blood oxygen saturation estimation can be based on the following general equation:

$${S}_{p} {O}_{2} = \frac{{HbO}_{2}}{{HbO}_{2} + {H}_{b}} , \qquad ( 1 )$$

whereas PPG devices attempt to “sense” HbO₂ and Hb via indirect non-invasive measurements.

Recently, approaches for remote photoplethysmographic oxygen saturation measurements have been presented. In this context, WO 2014/080313 A1 is referred to. The present disclosure generally seeks to detect new fields of application for rPPG-based devices.

In yet another embodiment of the device, each of the first combined signal and the second combined signal comprises a ratio of a first selected signal selected from the plurality of PPG signals and a second selected signal selected from the plurality of PPG signals.

This embodiment may be further developed in that each of the first combined signal and the second combined signal comprises a ratio of a time variant component of the first selected signal selected from the plurality of PPG signals and a time variant component of the second selected signal selected from the plurality of PPG signals. Assuming that the device makes use of three distinct PPG signals, the following equations may apply:

$$\begin{matrix}{RR}_{1} = \frac{A {C}_{1} / D {C}_{1}}{A {C}_{2} / D {C}_{2}} , & ( 2 ) \\ {SpO}_{2 \_ 1} = {C}_{11} - {C}_{12} {RR}_{1} , & ( 3 ) \\ {RR}_{2} = \frac{A {C}_{2} / D {C}_{2}}{A {C}_{3} / D {C}_{3}} , and & ( 4 ) \\ {SpO}_{2 \_ 2} = {C}_{21} - {C}_{22} {RR}_{2} , & ( 5 )\end{matrix}$$

wherein ACₓ is a respective time-variant pulsatile component of the respective PPG signal, wherein DCₓ is the respective non-pulsatile component of the respective PPG signal, wherein RR₁ and RR₂ are the respective ratio based on which the first combined signal and the second combined signal may be computed, and wherein Cₓ₁ and Cₓ₂ are the respective calibration constants for the calculation of the first combined signal and the second combined signal.

Assuming that the device makes use of four distinct PPG signals indicative of four distinct wavelength ranges, the following equations may apply:

$$\begin{matrix}{RR}_{1} = \frac{A {C}_{1} / D {C}_{1}}{A {C}_{2} / D {C}_{2}} , & ( 6 ) \\ {SpO}_{2 \_ 1} = {C}_{11} - {C}_{12} {RR}_{1} , & ( 7 ) \\ {RR}_{2} = \frac{A {C}_{3} / D {C}_{3}}{A {C}_{4} / D {C}_{4}} , and & ( 8 ) \\ {SpO}_{2 \_ 2} = {C}_{21} - {C}_{22} {RR}_{2} . & ( 9 )\end{matrix}$$

In yet another embodiment, the first combined signal is dependent on a tissue hydration level, wherein at least one PPG signal of the second combined signal is more dependent on a tissue hydration level than the PPG signals of the first combined signal. Preferably, the hydration level dependency of the second combined signal is at least increased by a factor of 2.0, more preferably by a factor of 5.0. In other words, the plurality of PPG signals, from which the first combined signal and the second combined signal may be calculated, may be selected such that considerable small changes of the hydration level may cause considerably large differences between the first combined signal and the second combined signal. Consequently, the device can be very sensitive to changes in the hydration state.

In yet another embodiment, the device further comprises a sensor unit, particularly an imaging unit, for remotely capturing image data, wherein the sensor unit is further configured to provide a data stream, the data stream comprising a plurality of PPG signals including at least a first PPG signal indicative of a first wavelength range, a second PPG signal indicative of a second wavelength range, and a third PPG signal indicative of a third wavelength range, wherein the PPG signals represent respective wavelength ranges derivable from the captured image data. By way of example, the sensor unit may be arranged as a video camera capable of capturing video data. Video data may cover visible radiation but also, at least partially, infrared radiation. Generally, the sensor unit may be configured to capture (video) image data in a considerably broad wavelength range from which the respective PPG signals may be selected.

However, at least in some embodiments, the device may comprise a contact sensor unit that can be attached to the subject's skin. For instance, the contact sensor unit may comprise at least three distinct contact sensors (e.g., photodiodes) each of which is configured to operate at a distinct wavelength range. Furthermore, at least one contact sensor may be envisaged that is capable of operating at three distinct wavelength ranges. The respective contact sensors may be coupled to or provided with respective radiation emitting elements, or, more precisely, light sources, such as light emitting diodes, etc. Also in a contact measurement environment, profit can be drawn from at least some aspects of the present disclosure.

In yet another aspect of the present invention, an unobtrusive PPG monitoring method for determining a hydration state of skin tissue is presented, the method comprising the following steps:
receiving a data stream comprising a plurality of PPG signals comprising at least a first PPG signal indicative of a first wavelength range, a second PPG signal indicative of a second wavelength range, and a third PPG signal indicative of a third wavelength range,
computing at least a first combined signal based on a first pair of signals selected from the plurality of PPG signals, and a second combined signal based on a second pair of signals selected from the plurality of PPG signals, wherein at least one PPG signal of the second combined signal is dependent on a tissue hydration level, and
computing a hydration signal indicative of skin hydration, wherein the hydration signal is derived from the first combined signal and a second combined signal.

Preferably, but not to be understood in a limiting sense, the method is arranged as an unobtrusive remote PPG monitoring method. Consequently, the method may be arranged as a non-contact PPG monitoring method. However, at least in some embodiments, the method can be arranged as a contact PPG monitoring method that utilizes at lest one contact PPG sensor.

In yet another aspect of the present invention, there is provided a computer program comprising program code means for causing a computer to perform the steps of the above method when said computer program is carried out on the computer.

As used herein, the term “computer” stands for a large variety of data processing devices. In other words, also medical devices and/or mobile devices having a considerable computing capacity can be referred to as computing device, even though they provide less processing power resources than standard desktop computers. Furthermore, the term “computer” may also refer to a distributed computing device which may involve or make use of computing capacity provided in a cloud environment.

Preferred embodiments of the invention are defined in the dependent claims. It should be understood that the claimed methods and the claimed computer program can have similar preferred embodiments as the claimed device and as defined in the dependent device claims.

### BRIEF DESCRIPTION OF THE DRAWINGS

These and other aspects of the invention will be apparent from and elucidated with reference to the embodiments described hereinafter. In the following drawings

FIG. 1 shows a schematic illustration of a general layout of a device in accordance with the present invention;

FIG. 2 shows a diagram illustrating a spectral absorption behavior of skin tissue components;

FIG. 3 shows a further diagram illustrating a spectral absorption behavior of skin tissue components;

FIG. 4 shows a schematic block diagram illustrating signal processing aspects of the present disclosure;

FIG. 5 shows a further schematic block diagram illustrating signal processing aspects of the present disclosure; and

FIG. 6 shows an illustrative block diagram representing several steps of an embodiment of a method in accordance with the present disclosure.

### DETAILED DESCRIPTION OF THE INVENTION

The following section describes exemplary approaches to photoplethysmography, in particular to remote blood oxygen saturation measurement, utilizing several aspects of the device and method of the invention. It should be understood that single steps and features of the shown approaches can be extracted from the context of the respective overall approach or embodiment. These steps and features can therefore be part of separate embodiments still covered by the scope of the invention.

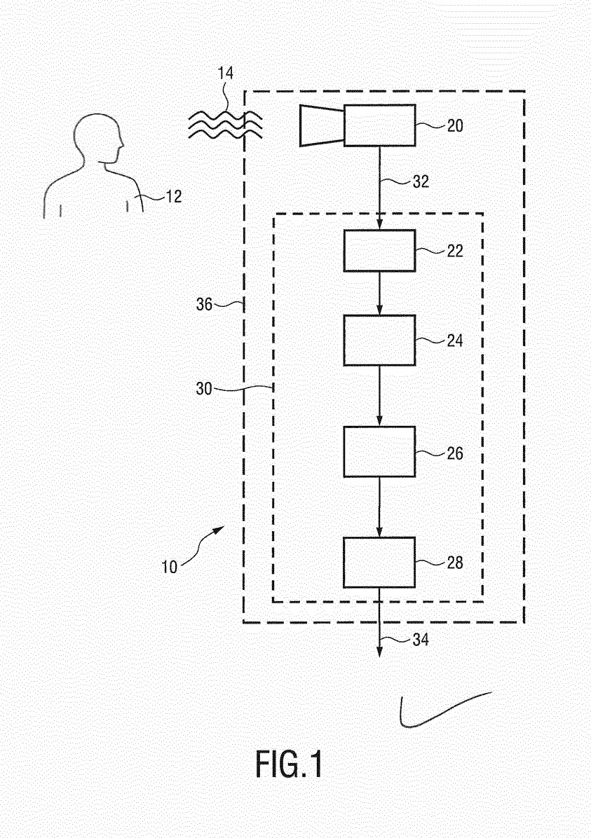

FIG. 1 shows a schematic illustration of a device for extracting physiological information which is denoted by a reference numeral 10. For instance, the device can be utilized for recording image frames representing a remote subject 12 or at least a portion of the subject 12 for remote PPG monitoring. In this connection, a region of interest in the subject 12 can be addressed when monitoring. The region of interest can comprise, by way of example, a forehead portion, a face portion or, more generally, a skin portion of the subject 12. The recorded data, for instance, a series of image frames, can be derived from electromagnetic radiation 14 reflected (or re-emitted) by the subject 12. Possibly, under certain conditions, at least part of the electromagnetic radiation could be emitted or transmitted by the subject 12 itself. Radiation transmission may occur when the subject 12 is exposed to strong illumination sources shining through the subject 12. Radiation emission may occur when infrared radiation caused by body heat is addressed and captured. However, for remote PPG applications, a huge portion of the electromagnetic radiation 14 to be captured can be considered radiation reflected by the subject 12. The subject 12 can be a human being or an animal, or, in general, a living being. Furthermore, the subject 12 can be considered as a part of a human being or an animal highly indicative of a desired signal.

A source of radiation, such as sunlight or an artificial radiation source, also a combination of several radiation sources can affect or impinge on the subject 12. The radiation source(s) basically emit(s) incident radiation striking the subject 12. Generally, the radiation source may be integrated in the device 10. However, in the alternative, the device 12 can also make use of external radiation sources. Generally, the device 10 may be configured to detect and/or process visible radiation and, furthermore, infrared radiation.

For extracting physiological information from respectively captured data, for instance, a sequence of image frames, a defined part or portion of the subject 12 such as a region of interest can be detected by a sensor 20. The sensor 20 can be embodied by an optical sensor adapted for capturing information belonging to at least one spectral component of the electromagnetic radiation 14. In a fairly simple embodiment, the sensor 20 may be embodied by a camera or a set of cameras.

Needless to say, the device 10 can also be adapted to process input signals, namely an input data stream 32, already recorded in advance and, in the meantime, stored or buffered. As indicated above, the electromagnetic radiation 14 can contain continuous or discrete characteristic signals, the PPG signals, which can be highly indicative of at least one vital parameter. The characteristic signal can be embodied in the input data stream 32.

Generally, the characteristic PPG signal is considered to contain a considerably constant (DC) portion and an alternating (AC) portion superimposing the DC portion. Applying signal processing measures, the AC portion can be extracted and, furthermore, compensated for disturbances. For instance, the AC portion of the characteristic signal can comprise a dominant frequency which can be highly indicative of the subject's 12 vascular activity, in particular the heart beat. Still, the characteristic signal, in particular the AC portion, can be indicative of further vital parameters. In this connection, the detection of blood oxygen saturation is an important field of application. The present disclosure primarily describes extended applications for PPG-based or, more precisely, rPPG-based methods and devices.

As indicated above, basically, blood oxygen saturation-representative values can be computed taking into account the behavior of the AC portion of the characteristic signal at distinct spectral portions thereof. In other words, a degree of blood oxygen saturation can be reflected in different radiation absorbance at blood vessels. Furthermore, one can make use of the fact that the difference in absorbance due to the grade of oxygenation also varies significantly across different spectral portions. Typically, the DC component represents the overall light absorption of the tissue, venous blood, and non-pulsatile arterial blood. By contrast, the AC component may represent the pulsatile arterial blood's absorption. Consequently, the determination of blood oxygen saturation (S_{P}O₂) can be expressed as:

$${S}_{p} {O}_{2} = C \cdot \frac{{( A C / D C )}_{red}}{{( A C / D C )}_{infrared}} , \qquad ( 10 )$$

where C is a calibration parameter (or represent a set of calibration parameters). C may stand for a large variety of calibration parameters applicable to the AC/DC relationship and should therefore not be interpreted in the strict algebraic sense of equation (10). Typically, in prior art measurement devices, C represents a fixed constant value, or a set of fixed constants.

By way of example, another exemplary S_{P}O₂ derivation model can be expressed as:

$${S}_{p} {O}_{2} = {C}_{1} + {C}_{2} \cdot \frac{{( A C / D C )}_{red}}{{( A C / D C )}_{infrared}} , \qquad ( 11 )$$

where C₁ and C₂ can be considered calibration parameters of a linear approximation. In an exemplary embodiment, the signal calibration parameter determination can be directed to adjust or adapt the parameter C₁. Still, in the alternative, S_{P}O₂ derivation may also be based on value tables deposited in (or accessible by) the device 10. The value tables (or: data bases) may provide for a discrete representation of the relationship between detected PPG signals and the desired calibration parameter. Also in that case an adaptable calibration parameter may be applied to improve the accuracy of the vital parameter determination.

It should be understood that the equations (10) and (11) are primarily presented for illustrative purposes. They should not be construed as limiting the scope of the present disclosure. It may be advantageous to combine S_{P}O₂ measurements and skin tissue hydration measurements. However, it may be also envisaged in the alternative that the skin tissue hydration determination is based on models and equations that are not applicable to S_{P}O₂ determination. In practice, a person skilled in the art may determine and establish further appropriate derivation models based on which the skin tissue hydration determination is may be established.

The data stream 32 comprising the continuous or discrete characteristic signal can be delivered from the sensor means 20 to an interface 22. Needless to say, also a buffer means could be interposed between the sensor means 20 and the interface 22. Downstream of the interface 22, the input data stream 32 can be delivered to a data processing module 30. The data processing module 30 can be considered a computing device, or at least, part of a computing device driven by respective logic commands (program code) so as to provide for desired data processing. The data processing module 30 may comprise several components or units which are addressed in the following.

It should be understood that each component or unit of the data processing module 30 can be implemented virtually or discretely. For instance, the data processing module 30 may comprise a number of processors, such as multi-core processors or single-core processors. At least one processor can be utilized by the data processing module 30. Each of the processors can be configured as a standard processor (e.g., central processing unit) or as a special purpose processor (e.g., graphics processor). Hence, the data processing module 30 can be suitably operated so as to distribute several tasks of data processing to adequate processors.

In accordance with an advantageous embodiment, the data processing module 30 is provided with or coupled to the interface 22 for receiving the data stream 32. The data stream 32 may comprise a plurality of PPG signals including at least a first PPG signal indicative of a first wavelength range, a second PPG signal indicative of a second wavelength range, and a third PPG signal indicative of a third wavelength range. Also a fourth PPG signal may be provided in the data stream 32. The respective PPG signals can be embedded in and extracted from the data stream 32. The data stream may 32 comprise video data comprising a representation of the subject 12.

The data processing module 30 may further comprise a processing unit 24 for computing at least a first combined signal based on a first pair of signals selected from the plurality of PPG signals, and a second combined signal based on a second pair of signals selected from the plurality of PPG signals. Preferably, at least one PPG signal of the first or the second combined signal is dependent on a present tissue hydration level, and the PPG signals of the other combined signal are not dependent on a present tissue hydration level, namely hydration invariant. This is attributable to the observation that water depositions in the skin tissue influence the light absorbance behavior with a wavelength-dependent intensity.

The data processing module 30 may further comprise an analysis unit 26 for computing a hydration signal 34 indicative of skin hydration, wherein the hydration signal 34 is derived from the first combined signal and the second combined signal. The hydration signal 34 may be provided or outputted via a respective output interface 28. In the alternative, the device 10 itself may comprise a display or similar output units.

A potential overall system boundary of the device 10 is denoted in FIG. 1 by a reference numeral 36. For instance, reference numeral 36 may stand for a common processing apparatus or housing. It should be understood that the device 10 can also be implemented as a distributed device.

It has been observed that an actual hydration state of the subject's skin tissue influences the absorption behavior of the skin tissue. Consequently, an actual degree of skin hydration (water disposed in skin tissue) is reflected in the data captured by the sensor 20 or, more generally, the device 10.

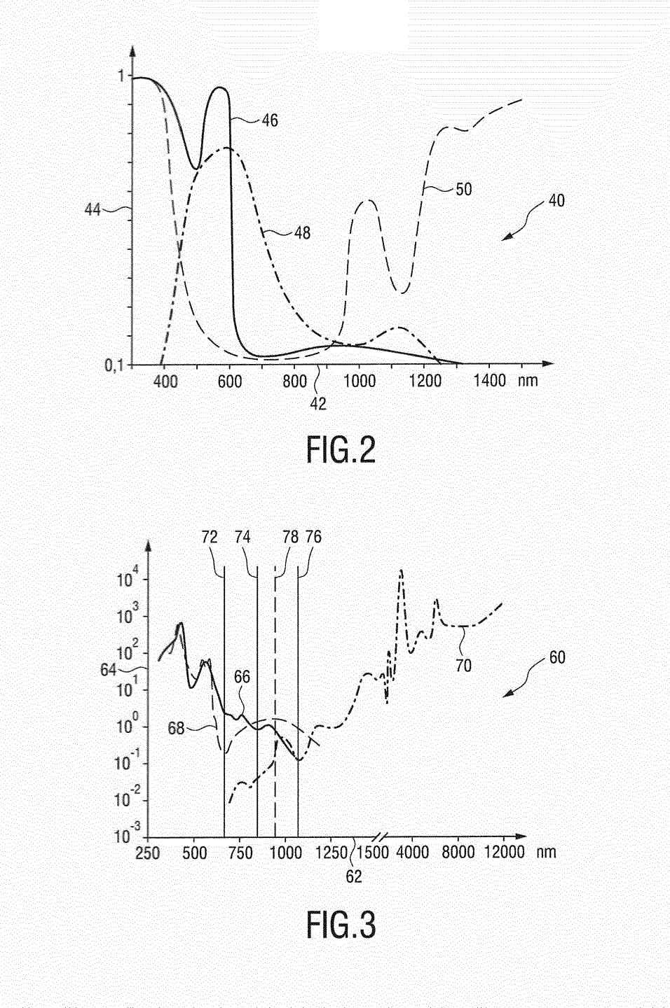

In this context, FIG. 2 and FIG. 3 illustrate a spectral dependency of the absorption behavior of several substances that may be present in skin tissue. FIG. 2 illustrates a qualitative light absorption diagram 40. FIG. 3 illustrates a quantitative light absorption diagram 60.

In FIG. 2, an axis of abscissas 42 represents an actual wavelength. Further, an ordinate axis 44 represents an actual absorption degree which may take values between 0 (no absorption at all) and 1 (total absorption). In FIG. 3, an axis of abscissas 62 illustrates a wavelength range. An ordinate axis 64 illustrates an absorption coefficient (unit [cm⁻¹]) which may take values between 10⁻³ and 10⁴ (logarithmic scale).

In FIG. 2, a graph 46 illustrates the spectral dependence of light absorption in hemoglobin. A graph 48 illustrates the spectral dependence of light absorption in melanin. A graph 50 illustrates the spectral dependence of light absorption in water. In FIG. 3, a graph 66 illustrates the spectral dependence of light absorption in hemoglobin (also referred to as Hb). The graph 66 relates to deoxygenated hemoglobin. By contrast, a graph 68 illustrates the spectral dependence of light absorption in oxygenated hemoglobin (also referred to as HbO₂). Accordingly, a graph 70 illustrates the spectral dependence of light absorption in water 70.

Depending on the wavelength of incident radiation and the actual composition of the skin tissue, a respective portion of the incident radiation will be absorbed. Consequently, a remaining portion of the incident radiation may be reflected (and/or transmitted) which can be detected by the sensor 20 and evaluated by the device 10.

As indicated in FIG. 3 by reference numerals 72, 74, 76, and 78 (as an option), a plurality of respective PPG signals indicating respective wavelength portions may be observed and/or obtained from image/video data so as to eventually derive a hydration-indicative signal therefrom. By way of example, a first PPG signal may be indicative of a wavelength or wavelength range 72 in the region of about 660 nm. A second PPG signal may be indicative of a wavelength or wavelength range 74 in the region of about 810 nm. A third PPG signal may be indicative of a wavelength or wavelength range 76 in the region of about 1050 nm. A fourth PPG signal may be indicative of a wavelength or wavelength range 78 in the region of about 910 nm.

As explained above, respective pairs of PPG signals may be selected from the plurality of PPG signals so as to process combined signals that may be—at least to some extent—attributable to oxygen saturation. An important aspect is that two respective combined signals can be obtained from the at least three distinct PPG signals. As a matter of fact, an oxygen saturation-indicative signal should basically have the same level (in terms of oxygenation) for both combined signals. Assuming that a considerable signal discrepancy can be detected, conclusions as to the actual water deposition in the observed skin tissue can be drawn therefrom. It may be therefore beneficial to select the plurality of PPG signals and the respective plurality of wavelength ranges 72, 74, 76, and 78 such that radiation absorption which is attributable to water depositions impacts the overall absorption at the respective ranges 72, 74, 76, and 78 to a varying degree.

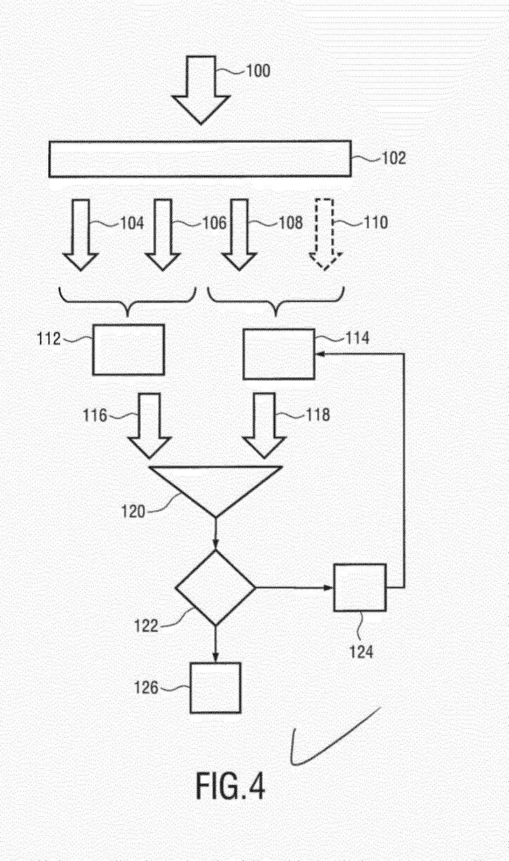

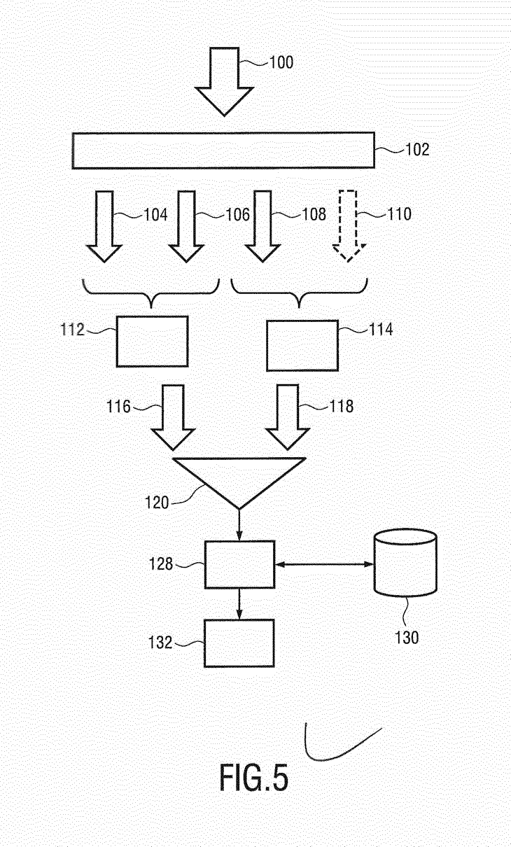

FIG. 4 and FIG. 5 illustrate signal processing approaches that may be utilized in a device 10 in accordance with the present disclosure.

As can be seen in both FIG. 4 and FIG. 5, a data stream 100 (which may correspond to the data stream 32 indicated in FIG. 1) may be received and processed. The data stream 100 preferably comprises a sequence of image frames. At least in some embodiments, the data stream 100 can be regarded as video data stream. The data stream 100 typically comprises a representation of a region of interest in the observed subject 12, typically a skin region. Furthermore, as illustrated by block arrows 104, 106, 108, and, optionally, 110, respective spectral sub-portions may be obtained from the data stream 100. To this end, a filter 102 may be utilized. The filter 102 may take the form of a hardware filter. Additionally, or in the alternative, the filter 102 may take the form of a software filter. The filter 102 may be generally arranged as a band filter. The filter 102 may be implemented at the level of the sensor 20 and/or the level of the data processing module 30. In the alternative, the sensor 20 may be configured to capture distinct PPG signals 104, 106, 108, 110 via a respective number of wavelength sensitive sensor elements and data transfer channels. Generally, each of the PPG signals 104, 106, 108, 110 may represent a considerably narrow sub-band of the electromagnetic spectrum, particularly in the visible light band and the infrared band.

As indicated by illustrative blocks 112, 114 in FIG. 4 and FIG. 5, combined signals 116, 118 may be computed based on the plurality of PPG signals 104, 106, 108, and 110. This may be performed by the processing unit 24. Each of the combined signals 116, 118 may be based on a pair of PPG signals selected from the plurality of PPG signals 104, 106, 108, 110. Consequently, at least three PPG signals are required so as to allow computing of the two distinct combined signals 116, 118. In some embodiments, four distinct PPG signals 104, 106, 108, and 110 are utilized such that each of the combined signals 116, 118 may be based on a distinct pair of distinct signals.

As further illustrated by reference number 120, the combined signals 116, 118 may be compared. To this end, a hardware and/or software signal comparator may be utilized. The signal comparison may be performed by the analysis unit 26. The signal comparison 120 may output a detected discrepancy between the first combined signal 116 and the second combined signal 118. The detected signal discrepancy may be utilized for an assessment of an actual hydration state of the observed skin tissue.

In this context, further reference is made to FIG. 4. In accordance with the embodiment illustrated in FIG. 4, some kind of “control loop” may be established. As indicated by reference numeral 122, a signal match between the first combined signal 116 and the second combined signal 118 may be performed. Assuming that a respective significant discrepancy is detected at 120 and 122, a respective modification of parameters for the calculation of the second combined signal 118 (and/or the first combined signal 116) may be performed as indicated by reference number 124. Hence, a further signal comparison may follow at 120 which may still result in a detected signal discrepancy between the first combined signal 116 and the second combined signal 118. However, eventually a signal match of the first combined signal 116 and the second combined signal 118 may be detected, assuming that the calculation parameters have been properly modified at 124. As illustrated by reference number 126, then an output signal may be generated that is highly indicative of an actual hydration state of the observed skin tissue. The output signal can be derived from the modified “calibration” parameters that enable the desired signal match of the first combined signal 116 and the second combined signal 118.

The skin tissue hydration detection approach illustrated in FIG. 5 is slightly modified. As illustrated by reference number 128, a present difference or discrepancy between the first combined signal 116 and the second combined signal 118 detected at 120 may be utilized to inspect a respective database 130 which may be embodied by a look-up table, for instance. Consequently, it is not required to modify respective parameters so as to eventually match the first combined signal 116 and the second combined signal 118. By contrast, a detected difference may serve as input value. Based on this input value, a desired output value that is indicative of the skin tissue hydration state may be obtained from the database 130 and eventually provided as a result, which is illustrated by a respective block indicated by reference number 132 in FIG. 5.

The above approaches illustrated in FIGS. 4 and 5 may be utilized for instantaneous measurements of a skin hydration state. However, at least in some embodiments, the above approaches may be utilized for continuous and/or quasi-continuous measurements of the skin hydration state. It may be further envisaged to calculate absolute skin hydration-indicative values which may require respective reference measurements. However, at least in some embodiments, it may be preferred to calculate relative skin hydration-indicative values which may allow trend monitoring, for instance.

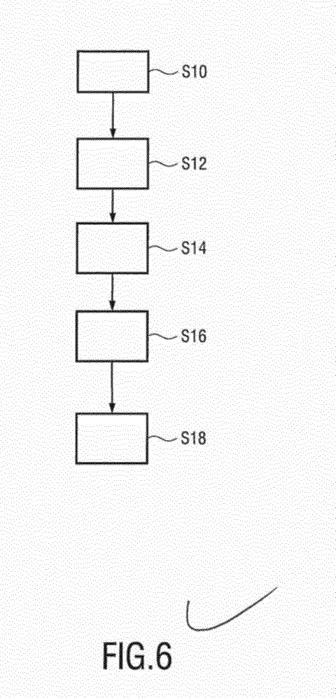

Having demonstrated several alternative exemplary approaches covered by the present disclosure, FIG. 6 is referred to, schematically illustrating a method for determining a hydration state of skin tissue which is preferably arranged as an unobtrusive remote PPG monitoring method. Initially, in a step S10 image data may be recorded or captured. Image data may comprise a sequence of (visible and near-infrared) image frames. Image data may be regarded as (visible and near-infrared) video data. At a further step S12 a data stream which is based on the captured data may be received. The data stream may comprise a plurality of PPG signals each of which is indicative of a respective wavelength range. Generally, it is preferred that at least three distinct PPG signals each of which is indicative of a respective wavelength range may be derived from the data stream.

At a further step S14, a first combined signal and a second combined signal may be computed. Each of the first combined signal and the second combined signal may be based on a respective pair of signals that are selected from the plurality of PPG signals that are embedded in the data stream received at step S12. As explained above, a skin tissue hydration level basically influences the light absorption behavior of the skin tissue in a wavelength-dependent manner. Consequently, also the transmittance and/or reflectance behavior is influenced which is present in the information provided in the data stream. Since light absorption of water is further dependent on an actual wavelength of the incident light and since the first combined signal and the second combined signal make use of PPG signals that represent distinct wavelength portions, a characteristic discrepancy between the first combined signal and the second combined signal may be detected and processed so as to draw a signal therefrom that is highly indicative of an actual skin tissue hydration level. The respective computation of the skin tissue hydration signal may be performed at a step S16. At a further step S18, a resulting hydration signal may be displayed, outputted and/or provided for further processing steps.

By way of example, the present invention can be applied in the field of health care, e.g. unobtrusive remote patient monitoring, general surveillances, security monitoring and so-called lifestyle environments, such as fitness equipment, or the like. Applications may include monitoring of oxygen saturation (pulse oximetry), heart rate, blood pressure, cardiac output, changes of blood perfusion, assessment of autonomic functions, and detection of peripheral vascular diseases which can be combined with the skin tissue hydration detection. Needless to say, in an embodiment of the method in accordance with the invention, several of the steps described herein can be carried out in changed order, or even concurrently. Further, some of the steps could be skipped as well without departing from the scope of the invention.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable (non-transitory) medium, such as an optical storage medium or a solid-state medium supplied together with or as part of other hardware, but may also be distributed in other forms, such as via the Internet or other wired or wireless telecommunication systems. Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible apparatus that can contain, store, communicate, propagate, or transport the program for use by or in connection with the instruction execution device.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and an optical disk. Optical disks may include compact disk—read only memory (CD-ROM), compact disk—read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

Any reference signs in the claims should not be construed as limiting the scope.

1. An unobtrusive PPG monitoring device for determining a hydration state of skin tissue, the device comprising:
an interface for receiving a data stream comprising a plurality of PPG signals including at least a first PPG signal indicative of a first wavelength range, a second PPG signal indicative of a second wavelength range, and a third PPG signal indicative of a third wavelength range,
a processing unit for computing at least a first combined signal based on a first pair of signals selected from the plurality of PPG signals, and a second combined signal based on a second pair of signals selected from the plurality of PPG signals, wherein at least one PPG signal of the first or second combined signal is dependent on a tissue hydration level and considerably sensitive to water deposition in the monitored skin tissue, and the PPG signals of the other combined signal are insensitive to water deposition in the monitored skin tissue;
an analysis unit for computing a hydration signal indicative of skin hydration, wherein the hydration signal is derived from the first combined signal and the second combined signal.

2. The device as claimed in claim 1, wherein the analysis unit is further configured to match the first combined signal and the second combined signal, and to compute the hydration signal based on the signal match.

3. The device as claimed in claim 2, wherein the analysis unit is further configured to compute the hydration signal under consideration of a predefined set of calibration constants for the first combined signal by revising variable calibration constants for the second combined signal.

4. The device as claimed in claim 1, wherein the analysis unit is further configured to monitor relative changes of the hydration signal over time in a continuous or quasi-continuous manner.

5. The device as claimed in claim 1, wherein the processing unit is further configured to compute the at least first combined signal under consideration of a first predefined set of calibration constants and to compute the at least second combined signal under consideration of a second predefined set of calibration constants, and wherein the analysis unit is further configured to compute the hydration signal based on reference values for skin hydration levels and a detected discrepancy between the at least first combined signal and the at least second combined signal.

6. The device as claimed in claim 5, wherein the analysis unit is further configured to monitor absolute tissue hydration level values based on a data set of reference values obtained from reference measurements.

7. The device as claimed in claim 1, wherein the first wavelength range is selected from the red wavelength range, wherein the second wavelength range is selected from the near infrared wavelength range, wherein the third wavelength range is selected from a deeper infrared wavelength range, and wherein the third wavelength range comprises larger wavelengths than the second wavelength range.

8. The device as claimed in claim 7, wherein the plurality of PPG signals further comprises at least a fourth PPG signal indicative of a fourth wavelength range, wherein the fourth wavelength range is selected from a the wavelength portion spanning the red wavelength band and the near infrared wavelength band, preferably, the fourth wavelength range is selected from a deeper infrared wavelength range.

9. The device as claimed in claim 1, wherein each of the first combined signal and the second combined signal is an oxygen saturation indicative signal.

10. The device as claimed in claim 1, wherein each of the first combined signal and the second combined signal comprises a ratio of a first selected signal selected from the plurality of PPG signals and a second selected signal selected from the plurality of PPG signals.

11. The device as claimed in claim 10, wherein each of the first combined signal and the second combined signal comprises a ratio of a time variant component of the first selected signal selected from the plurality of PPG signals a time variant component of the second selected signal selected from the plurality of PPG signals.

12. The device as claimed in claim 1, wherein the first combined signal is dependent on a tissue hydration level, wherein at least one PPG signal of the second combined signal is more dependent on a tissue hydration level than the PPG signals of the first combined signal, preferably increased by a factor of at least 2.0, more preferably by a factor of 5.0.

13. The device as claimed in claim 1, further comprising a sensor unit, particularly an imaging unit, for remotely capturing image data, wherein the sensor unit is further configured to provide a data stream, the data stream comprising a plurality of PPG signals including at least a first PPG signal indicative of a first wavelength range, a second PPG signal indicative of a second wavelength range, and a third PPG signal indicative of a third wavelength range, wherein the PPG signals represent respective wavelength ranges derivable from the captured image data.

14. An unobtrusive PPG monitoring method for determining a hydration state of skin tissue, the method comprising the following steps:
receiving a data stream comprising a plurality of PPG signals including at least a first PPG signal indicative of a first wavelength range, a second PPG signal indicative of a second wavelength range, and a third PPG signal indicative of a third wavelength range,
computing at least a first combined signal based on a first pair of signals selected from the plurality of PPG signals, and a second combined signal based on a second pair of signals selected from the plurality of PPG signals, wherein at least one PPG signal of the first or second combined signal is dependent on a tissue hydration level and considerably sensitive to water deposition in the monitored skin tissue, and the PPG signals of the other combined signal are insensitive to water deposition in the monitored skin tissue;
computing a hydration signal indicative of skin hydration, wherein the hydration signal is derived from the first combined signal and the second combined signal.

15. A computer program comprising program code means for causing a computer to carry out the steps of the method as claimed in claim 14 when said computer program is carried out on a computer.
